(19) United States
(12) Patent Application Publication
(10) Pub. No.: US 2026/0227649 A1
(43) Pub. Date: Aug. 6, 2026

(54) A LIQUID LENS, A CONTACT LENS CONTAINING THE LIQUID LENS, AND AN INTRAOCULAR DEVICE

(71) Applicant: XPANCEO RESEARCH ON NATURAL SCIENCE L.L.C., Dubai (AE)

(72) Inventors: Aleksey Vladimirovich Arsenin, Moscow (RU); Vyacheslav Sergeevich Brunov, Moscow (RU); Valentin Sergeevich Volkov, Dolgoprudnyy (RU); Georgii Alekseevich Ermolaev, Dolgoprudnyy (RU)

(21) Appl. No.: 19/151,314

(22) PCT Filed: Feb. 28, 2024
(86) PCT No.: PCT/RU2024/050046

§ 371 Date: Jul. 26, 2025

(30) Foreign Application Priority Data
Jan. 31, 2023 (RU) 2023102019

(51) Int. Cl. G02C 7/04

(52) U.S. Cl. CPC G02C 7/049

(57) ABSTRACT

The group of inventions relates to the optical elements with variable characteristics, and specifically to the liquid lenses with variable optical power, and the contact lenses with enhanced functionality that are equipped with such liquid lenses. The liquid lens is implemented as a capsule containing control electrodes, immiscible first liquid medium having the refractive index n₁ and second liquid medium having the refractive index n₂, such that n₂>n₁. One of said liquid media is adapted to be controlled by the electromagnetic field via said electrodes. The second liquid medium represents a colloidal system formed by a liquid and solid high-refractive nanoparticles whose refractive index is higher than the refractive index of said liquid. The invention makes it possible to expand the possible application areas of such liquid lens by expanding the range of achievable optical power. The contact lens equipped with the above described liquid lens in the first embodiment enables the implementation of the zoom function, in the second embodiment when used with a microdisplay implements an AR/VR/XR mode, in the third embodiment when used with light emitting diodes implements a myopia prevention and treatment function. The intraocular device equipped with the above described liquid lens makes it possible to replace the user's crystalline lens and in addition has an enhanced functionality.

### TECHNICAL FIELD

The group of inventions relates to the optical elements with variable characteristics, and specifically to the liquid lenses with variable optical power, and the contact lenses containing said liquid lenses, wherein said contact lenses can be used to create a magnified image of the objects within the field of view to form an augmented, virtual, or extended reality (AR/VR/XR), to prevent and treat myopia, and also to intraocular lenses.

### BACKGROUND ART

As is known, a human eye has a vertical angle of view of ~120° and a horizontal angle of view of ~180° (this is true for both eyes simultaneously, for achromatic vision), and the crystalline lens when focusing on the objects at different distances (eye accommodation) can change the eye (vitreous) back wall curvature and, accordingly, the focal distance, thereby changing the focusing distance. A healthy person's crystalline lens is able to change its curvature to enable the optical power variation approximately from 19 to 33 diopters. However, the human eye's optical system has no independent possibility to optically expand (the ratio between the linear or angular dimensions of the image and the object) the field of view without any additional external devices.

The optical magnification or the varying angular magnification of an optical system, i.e. the zoom function, can be implemented using such devices as varifocal lenses, zoom lenses, or variable focus distance lenses. The optical magnification in these conventional systems is achieved through variation of the focal distance by moving individual lens elements inside the lens carrier—the smaller the optical system focal distance, the larger the angle of view (field of view), and vice versa. Variation of the focal distance and, accordingly, of the field of view can be both stepwise and smooth (pancratic). Herewith, the number of the lens elements in such lens can exceed 10-20 pieces meaning that it cannot have a compact design, which makes it impossible to build it into a wearable device without a serious discomfort for a user.

The prior art discloses a liquid lens implemented as a capsule containing the immiscible first liquid medium having the refractive index n₁ and second liquid medium having the refractive index n₂, such that n₂>n₁, and the control electrodes, wherein one of said liquid media is adapted to be controlled by the electromagnetic field via said electrodes (see publication WO2020132443A2, cl. G02B 26/00, published 25 Jun. 2020). In the prior art device, the first liquid is an aqueous solution of one or several electrolytes, while the second liquid represents a silicone oil or alkanes, which makes it possible to achieve the maximum refractive index difference Δn=n₂−n₁ approximately 0.05. The optical power of such lens can vary depending on the curvature of said liquids' free meniscus, which in its turn is controlled by electromagnetic field, and varies from 0 (for a flat meniscus) to approximately 10 diopters. Such liquid lens as a reconfigurable optical system together with a power and control module is suggested to be integrated into a contact lens, which makes it possible to use the obtained device for vision correction. The main disadvantage of the prior art solutions is their very limited functionality: little difference in the refractive indices of simple (ordinary, common) liquids (not more than 0.1) only allows to vary a liquid lens optical power in a relatively small range, which limits its application by using only as an element that compensates the defects of natural accommodation, but not for a zoom function.

Another current problem is to create compact AR, VR, XR devices based on light emitting diode microdisplays.

The prior art discloses a contact lens for forming an augmented or extended reality, comprising a display with a screen directed towards the user's eye, a power and control module, and a telescopic collimating optical system disposed between the display and the eye (see patent U.S. Pat. No. 10,353,205B2, cl. G02C 7/04, published 16 Jul. 2019). The principle of operation of a collimating optical system is based on a multiple reflection of the rays of light: the image from the source (a display) in the form of divergent rays passes along the device chamber and reflects from the ‘secondary’ convex mirror, returns back and reflects from the ‘primary’ concave mirror whose focus is on the ‘secondary’ mirror. Thus, the image reflected from the ‘secondary’ mirror exits the chamber aperture as a collimated beam of light towards the user's eye that, in its turn, focuses the image on the retina.

Such femtoprojector optical system can form a magnified display image on the user's eye retina, and the system dimensions make it possible to build the display device into a contact lens. However, the main disadvantage of the prior art device is its relatively big thickness (approximately 2 mm), which makes its use rather uncomfortable. Furthermore, the prior art device is difficult in fabrication and adjustment of optical parts, has a limited field of view, and forms a nonuniform light pattern on the retina. A projector like this always has ‘parasitic’ peripheral rays from the source that do not hit the ‘secondary’ mirror and exit the chamber aperture as a divergent beam. Such rays do not focus on the retina, but come as divergent rays that results in optical aberrations and blurred spots in the background of the focused display image, and also in decreased resulting brightness of the image on the retina because a part of the rays has scattered.

Vision correction applications are another trend of expanding the functionality of the wearable optical devices in the form of contact lenses. The human eye has a natural ability to accommodate, i.e. to focus the sight on the outer world objects located at different distances from the observer. The contraction and relaxation of the eye ciliary muscle provide variation of the crystalline lens back wall radius and focusing the image on the retina.

One of the most common vision problems is myopia (shortsightedness). With this vision abnormality, a person has a good ability to distinguish objects at a short distance, but the eye is unable to clearly focus on distant objects (including those at infinity), due to which fact the distant objects appear blurred. Myopia as an abnormality of refraction (deflection) can occur as a result of the fact that the eye inherently has an oblong shape and hence the images of the distant objects even with completely relaxed crystalline lens are focused at some distance in front of the retina rather than on the retina itself. Also, myopia can occur due to prolonged screen time on a computer or constant use of electronic gadgets (when the crystalline lens remains in a state of tension for most of the day) or can be a result of genetic predisposition.

A known method to slow down, to prevent the development and even to partially cure myopia is the method referred to as myopic defocus, consisting in forming a ‘stimulus’ on the retina periphery, such as a defocused image. Such stimuli suppress the eye lengthwise growing and slow down or stop the development of myopia. The prior art discloses a device for preventing and treating myopia (shortsightedness) implemented as a contact lens comprising four light emitting diodes disposed in the contact lens peripheral part and directed towards the user's eye, a power and control module, and an optical system disposed between the light emitting diodes and the eye (see publication WO2021056018A1, cl. G02C 11/04, published 25 Mar. 2021). In the prior art device, said optical system is based on refractive elements (micromirrors) focusing the emission from the light emitting diodes close to the user's eye retina so that to cause the user's involuntary (unconscious) intention to focus the image of the resulting point (spot), which trains the eye muscles and makes it possible to prevent myopia and as well as treat it at early stages. The disadvantages of the prior art device are the necessity to account for individual peculiarities of the user's vision, the complexity of adjusting the refractive elements, and impossibility to regulate the degree of the light emitting diode image defocus in the course of treatment.

Yet another common defect of vision (more than 8% of population over 50 years of age) is cataract, i.e. a partial or complete clouding of the crystalline lens that leads to significant weakening of vision and to reversible blindness. To cure blindness caused by damage of a crystalline lens (for example, due to cataract, glaucoma, physical injury or the like), a surgical intervention is recommended to remove the injured crystalline lens and replace it, for example, with an intraocular lens. Once the natural crystalline lens is removed, a common monofocal or multifocal intraocular lens can be placed into the eye posterior chamber. The problem with such devices is the absence of natural ability to accommodate. Therefore, to accommodate on close objects, users of intraocular lenses have to use additional optical devices, such as glasses or contact lenses.

The prior art discloses an intraocular device comprising an intraocular lens adapted to accommodate in accordance with the user's control signals, and holding elements to fix said intraocular lens inside the user's eye (see patent U.S. Pat. No. 8,377,125B2, cl. A61F2/16, published 19 Feb. 2013). In the prior art device, accommodation is provided using the holding elements implemented as specific flexible tabs (referred to as haptics) which, using the tension of the eye ciliary muscle, can change the intraocular lens position along the eye optical axis, and the intraocular lens optical power. The disadvantage of this method is the necessity of a postsurgical period during which a ciliary muscle relaxant is periodically introduced into the eye—this is a healing period (from two to three weeks) to maintain the ciliary muscle in a relaxed state until fibrosis is complete. Medicated relaxation of the ciliary muscle prevents its contraction and immobilizes the capsule of lens. Another disadvantage is the physical movement along the eye optical axis depending on the ciliary muscle tension, which can be imperfect and may provide incomplete accommodation.

Thus, the technical problem is to eliminate said disadvantages of the prior art technology and to create compact wearable optical devices in the form of contact lenses and intraocular devices with expanded functionality.

### SUMMARY OF INVENTION

As related to the liquid lens design, the technical effect consists in significant expansion of its possible areas of application by expanding the range of achievable optical power. The set problem has been solved and the technical effect has been achieved by that in the liquid lens implemented as a capsule containing the immiscible first liquid medium having the refractive index n₁ and second liquid medium having the refractive index n₂, such that n₂>n₁, and the control electrodes, one of said liquid media is adapted to be controlled by the electromagnetic field via said electrodes, and the second liquid medium represents a colloidal system formed by a liquid and solid high-refractive nanoparticles whose refractive index is higher than the refractive index of said liquid. Said first liquid medium and second liquid medium are preferably implemented in such a way that the difference of their refractive indices Δn=n₂−n₁>1.5. The high-refractive nanoparticles can be made of ZnO, TiO₂, ZnS, MgO, BeO, PbF₂, CsI, HfO₂, Sc₂O₃, SiN, GaP, CsPbBr₃, CsPbCl₃, CsPbI₃, GaN, YVO₄, MgAlO, YAlO, LuAlO, AlSb, GaSb, InSb, AlAs, GaAs, InAs, BC, SiC, TiC, VC, CsCl, CuCl, BaF, CeF₃, LaF₃, LiF, SrF₂, LiI, KI, RbI, CaMoO₄, SrMoO₄, PbMoO₄, LiNbO₃, KNbO₃, VN, ZrO₂, GeO₂, TeO₂, WO₃, Fe₂O₃, Y₂O₃, Lu₂O₃, Nb₂O₅, Ta₂O₅, Fe₃O₄, InP, CdSe, PbSe, ZnSe, AgGaS₂, CdGa₂S₄, CdS, CuGaS₂, CdTe, Te, ZnTe, BaTiO₃, Bi₄Ti₃O₁₂, PbTiO₃, SrTiO₃, diamond or a van der Waals material consisting of two-dimensional layers bonded with each other by the van der Waals forces, for example, graphite, graphene, graphene oxide, MoS₂, WS₂, MoSe₂, WSe₂, Cd₃As₂, Cd₃Sb₂, Cr₂AlC, Cr₂C, Mn₂AlC, Mo₂C, Mo₂Ga₂C, Mo₃AlC₂, Nb₂AlC, Nb₂C, Nb₄AlC₃, Nb₄C₃, Ta₂C, Ta₄AlC₃, Ti₂AlC, Ti₂AlN, Ti₂C, Ti₂N, Ti₃AlC₂, Ti₃C₂, Ti₃CN, Ti₃SiC₂, TiAlN₃, V₂AlC, V₂C, V₄AlC₃, V₄C₃, SnS₂, SnSe₂, ReS₂, ReSe₂, hBN, GaSe, Sb₂Te₃, PdS₂, PdSe₂, PtS₂, PtSe₂, GaS, GaTe, Ca(OH)₂, K(FeMg)₃Si₃AlO₁₀(OH)₂, Mg(OH)₂, MnO₂, MoO₃, Sb₂O₃, Sb₂OS₂, Sb₂Se₃, Sb₂S₃, As₂S₃, As₂Te₃, Bi₂O₂Se, Bi₂Se₃, Bi₂TeO₂, BiSbTe₃, Bi₂S₃, Bi₂Te₃, AsP, CdI₂, CdPS₃, CuS, CoPS₃, Cr₂Ge₂Te₆, Cr₂S₃, CrBr₃, CrCl₃, CrGeTe₃, CrPS₄, CrSeBr, CuCrP₂S₆, CuIn₇Se₁₁, FeCl₂, FePS₃, FePSe₃, MoTe₂, GaGeTe, GalnS₃, GaSeTe, GaSSe, GaPS₄, GaSTe, GeAs, GeSe, GeS, GeS₂, GeTe, HfSe₂, HfS₂, HfTe₂, In₂S₃, In₂Se₃, InSe, InTe, InGaSe₂, InSeBr, InSnSe, MnPS₃, MnPSe₃, MoSSe, MoWSe₂, MoWS₂, MoWTe₂, MoNbSe₂, MoO_{2.5}Cl_{0.5}, MoReS₂, MoTaSe₂, MoVSe₂, Na₂Co₂TeO₆, Nb₂SiTe₄, NbReS₂, NbReSe₂, NbS₃, Ni₂SiTe₄, Ni₃TeO₆, NiCl₂, NiI₂, NiPS₃, PbI₂, PbTe, PtTe₂, ReMoS₂, ReNbS₂, ReNbSe₂, ReSSe, SbAsS₃, SbSe, SbSI, SiP, SnPSe₃, SnS, Ta₂NiS₅, TaS₂, TaS₃, TaSe₂, TaWSe₂, TlSe, TiBr₃, SnTe₂, TiS₃, TIGaS₂, TIGaSe₂, TIGaTe₂, TlInS₂, WTe₂, WSSe, WNbSe₂, WReSe₂, ZrS₂, ZnIn₂S₄, ZnPS₃, ZnPSe₃, ZrGeTe₄, ZrS₃, ZrSe₂, ZrSe₃, ZrTe₂, ZrTe₃, Cr₂Si, Te₆, Cr₂Te₃, CrI₃, CrSBr, CrTe₂, Fe₃GeTe₂, Fe₄GeTe₂, TaCo₂Te₂, VS₂, VSe₂, VTe₂BiSbTeSe, BiTe, CuFeTe, HfTe₅, FeSe, FeTeSe, FeTe, NbS₂, NbSe₂, NbTe₂, NbTe₄, NiTe₂, PdBi₂, PdTe₂, SnTaS₂, TaTe₂, TiTe₂, Tl₂Ba₂CaCu₂Og, ZrSiS, CdAs₂, CuSi₂P₃, NbAs₂, PbTaSe₂, Ta₂NiSes, Ta₂NiTes, Ta₂Se₈I, TaNi₂Te₃, TiS₂, TiSe₂, WNbTe₂, ZnAs₂, ZrTe₅, LaTe₂, NbSe₃, BizSeTe₂, Bi₂Te₂S, BiInTe₃, Bi₂Se_{1.5}Te_{1.5}, Bi₄Te_{1.5}S_{1.5}, GeBi₂Te₄, PbBi₂Te₄, SnBi₄Te₇, SnSb₂Te₄, NiTe, SbTe, SiTe₂, BiTeI, InSSe, PbSnS₂, TlGaS₃, C₃N₄, Cu₂Te, GeSeTe, MnTe, As₂Se₃, CrPS₃, SnSe, WReS₂, TiBr, BaTiS₃, Al₂O₃, BiFeO₃, Ag₃AsS₃, HgS, bismuth strontium calcium copper oxide, black arsenic, or black phosphorus.

As related to the contact lens design according to the first embodiment, the technical effect consists in expanding its functionality by realizing the function of magnifying the image of the objects in the field of view (a zoom function). The set problem has been solved and the technical effect has been achieved in this regard by that the contact lens contains a power and control module and a reconfigurable optical system equipped with the above suggested liquid lens occupying at least a part of the user's eye field of view and adapted to focus the images of the objects located within said part of the field of view on the user's crystalline lens, and to provide visible magnification of these objects. Herewith, the liquid lens optical power preferably comprises not less than 300 diopters and enables a magnification of the objects within the field of view of at least two times. The part of the field of view occupied by the liquid lens can comprise from 30 to 60%.

As related to the contact lens design according to the second embodiment, the technical effect consists in reducing the overall dimensions of a contact lens with an augmented, virtual, or extended reality (AR, VR, XR) function. The set problem has been solved and the technical effect has been achieved in this regard by that the contact lens contains a display with a screen directed towards the user's eye, a power and control module, and an optical system that is equipped with the above suggested liquid lens adapted to focus the emission from the screen on the user's crystalline lens. Herewith, the liquid lens optical power preferably comprises not less than 800 diopters. The liquid lens can be installed with a gap relative to the screen, wherein the gap is formed by a hollow chamber or a layer of polymer material.

As related to the contact lens design according to the third embodiment, the technical effect consists in expanding the range of possible therapeutic intervention by providing the possibility to vary the degree of defocus of the image formed on the user's eye retina. The set problem has been solved and the technical effect has been achieved in this regards by that the contact lens contains at least one light emitting diode disposed in the contact lens peripheral part and directed towards the user's eye, a power and control module, and an optical system equipped with the above suggested liquid lens adapted to focus the emission from the light emitting diode in front of the user's retina. Herewith, the liquid lens optical power comprises not less than 1000 diopters. The liquid lens is preferably adapted to vary the degree of defocus of the emission from the light emitting diode on the user's retina in the range from 0.5 to 10 diopters. Herewith, the contact lens can have from 2 to 40 light emitting diodes disposed in its peripheral part.

As related to the intraocular device, the technical effect consists in expanding its functionality, in particular, by enhancing the accommodation ability up to the level exceeding physiological capabilities of the human eye. The set problem has been solved and the technical effect has been achieved in this regards by that in the intraocular device containing an intraocular lens, adapted to accommodate in accordance with the user's control signals, and holding elements to fix said intraocular lens inside the user's eye, said intraocular lens is equipped with the above suggested liquid lens and the power and control module configured to convert the user's control signals into the electromagnetic field formed by the liquid lens control electrodes. The power and control module is preferably configured to vary the liquid lens optical power at least from 19 to 33 diopters, alternatively up to 200 diopters. Herewith, the power and control module is preferably configured to transmit an activation signal to the light emitting diodes in the contact lens when the liquid lens optical power has exceeded 35 diopters. The liquid lens capsule is preferably made of elastic polymer material, while the holding elements can be made integral with the liquid lens capsule. The holding elements can be implemented as tabs formed from elastic polymer material.

### BRIEF DESCRIPTION OF DRAWINGS

FIG. 1 is a cross-section view of the disclosed liquid lens with the electromagnetic field deactivated;

FIG. 2 is the same as FIG. 1, with the electromagnetic field activated to the maximum;

FIG. 3 is the contact lens according to the first embodiment with a 100% field of view coverage and the zoom function deactivated;

FIG. 4 is the same as FIG. 3, with the zoom function activated;

FIG. 5 is a diagram of forming the image through the contact lens according to the first embodiment with 100% field of view coverage and the zoom function deactivated;

FIG. 6 is the same as FIG. 5, with the zoom function activated;

FIG. 7 is the contact lens according to the first embodiment with 50% field of view coverage and the zoom function deactivated;

FIG. 8 is the same as FIG. 7, with the zoom function activated;

FIG. 9 is a diagram of forming a magnified image using the contact lens according to the first embodiment with 50% field of view coverage and the zoom function activated;

FIG. 10 shows the image visible by the eye when implementing the scheme according to FIG. 9;

FIG. 11 shows a projecting system based on a liquid lens arranged in relation to the display with an air gap formed by a hollow chamber;

FIG. 12 shows a projection system based on a liquid lens arranged in relation to the display with a gap formed by a layer of polymer material;

FIG. 13 is a diagram of forming an image using the contact lens according to the second embodiment with an AR/VR/XR function, cross-section view;

FIG. 14 shows the contact lens according to the second embodiment with an AR/VR/XR function, front view;

FIG. 15 is a diagram of forming an image using the contact lens according to the third embodiment with a myopia correction function with one light emitting diode installed;

FIG. 16 is a diagram of forming an image using the contact lens according to the third embodiment with several light emitting diodes installed;

FIG. 17 shows the contact lens according to the third embodiment with 16 light emitting diodes, front view;

FIG. 18-FIG. 20 show a general view of the disclosed intraocular device with various holding elements;

FIG. 21 shows installation of the intraocular device inside the user's eye in front of the iris;

FIG. 22 is a diagram of forming an image using the disclosed intraocular device installed behind the iris when accommodating at infinity (the liquid lens optical power is 19 diopters);

FIG. 23 is the same as FIG. 22, but when accommodating at a close physiological point (the liquid lens optical power is 33 diopters);

FIG. 24 is the same as FIG. 22-FIG. 23, but when realizing a macro vision mode (the liquid lens optical power is 138 diopters, thickness 1 mm).

### DESCRIPTION OF EMBODIMENTS

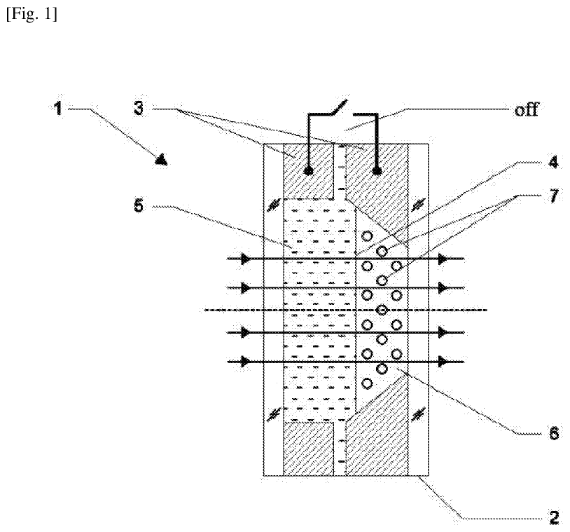

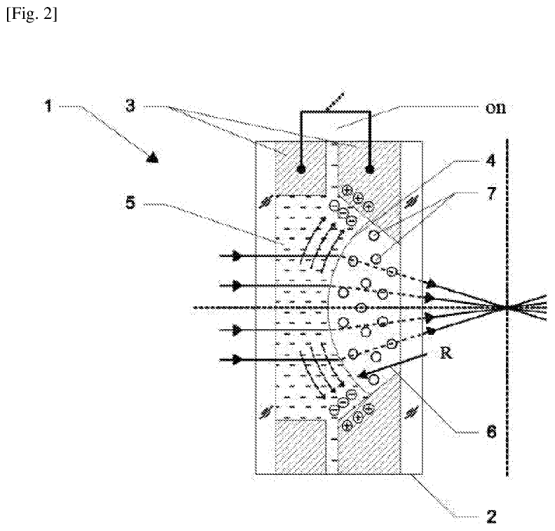

The suggested group of inventions implies using the design of the liquid lens 1 comprising the capsule 2 and the control electrodes 3 installed on the periphery FIG. 1-FIG. 2. The inlet and outlet windows of the capsule 2 are made of a material transparent in the visible range of the spectrum, such as glass, crystal, nano-glass ceramics, or an optically transparent polymer. The capsule 2 houses two immiscible (non-wettable, having different viscosity) liquid media with different refractive indices, one of which (any) is adapted to be controlled by the electromagnetic field via the electrodes 3. This makes it possible to control the curvature of the boundary surface between said liquid media, i.e. the meniscus 4. To enable such control, one of the media can contain magnetic nanoparticles and/or possess high electrical conductivity.

The first liquid medium 5 is, for example, water or a water solution having the refractive index n₁, which comprises from 1.2 to 1.4 in the visible range. The refractive index lower than that of water (1.33) is achieved by introducing porous nanoparticles made of a material with low refractive index and low extinction coefficient in the visible range of the spectrum (for example, SiO₂, TiN, HIN, ZrN, YN, VN, WN). Such nanoparticles can be synthesized using the method of femtosecond laser ablation in deionized water under the conditions inducing catalytic decomposition of water into hydrogen and oxygen. The lower is n₁, the higher is the maximum optical power that can be realized using the liquid lens 1.

The second liquid medium 6 represents a colloidal system having the refractive index n₂, such that n₂>n₁. This colloidal system is formed by a high-refracting immersion liquid, for example, a hydrophobic oil having the refractive index from 1.4 to 1.8 and solid high-refractive nanoparticles 7 having the size from 2 to 250 nm and a low extinction coefficient. The refractive index of the nanoparticles 7 in the visible range is higher than that of said immersion liquid and comprises from 1.8 to 3, preferably 3. The optical power of the obtained liquid lens 1 can be significantly expanded by realizing the refractive index difference Δn=n₂−n₁>1.5.

Such high-refractive material for the nanoparticles 7 can be represented by nonlaminar materials with a high refractive index and high transparency in the visible range, such as ZnO, TiO₂, ZnS, MgO, BeO, PbF₂, CsI, HfO₂, Sc₂O₃, SiN, GaP, CsPbBr₃, CsPbCl₃, CsPbI₃, GaN, YVO₄, MgAlO, YAlO, LuAlO, AlSb, GaSb, InSb, AlAs, GaAs, InAs, BC, SiC, TiC, VC, CsCl, CuCl, BaF, CeF₃, LaF₃, LiF, SrF₂, LiI, KI, RbI, CaMoO₄, SrMoO₄, PbMoO₄, LiNbO₃, KNbO₃, VN, ZrO₂, GeO₂, TeO₂, WO₃, Fe₂O₃, Y₂O₃, Lu₂O₃, Nb₂O₅, Ta₂O₅, Fe₃O₄, InP, CdSe, PbSe, ZnSe, AgGaS₂, CdGa₂S₄, CdS, CuGaS₂, CdTe, Te, ZnTe, BaTiO₃, Bi₄Ti₃O₁₂, PbTiO₃, SrTiO₃, or diamond. However, it appears that the most promising will be the use of the van der Waals materials consisting of two-dimensional layers bonded with each other by the van der Waals forces: graphite, graphene, graphene oxide, MoS₂, WS₂, MoSe₂, WSe₂, Cd₃As₂, Cd₃Sb₂, Cr₂AlC, Cr₂C, Mn₂AlC, Mo₂C, Mo₂Ga₂C, Mo₃AlC₂, Nb₂AlC, Nb₂C, Nb₄AlC₃, Nb₄C₃, Ta₂C, Ta₄AlC₃, Ti₂AlC, Ti₂AlN, Ti₂C, Ti₂N, Ti₃AlC₂, Ti₃C₂, Ti₃CN, Ti₃SiC₂, Ti₄N₃, V₂AlC, V₂C, V₄AlC₃, V₄C₃, SnS₂, SnSe₂, ReS₂, ReSe₂, hBN, GaSe, Sb₂Te₃, PdS₂, PdSe₂, PtS₂, PtSe₂, GaS, GaTe, Ca(OH)₂, K(FeMg)₃Si₃AlO₁₀(OH)₂, Mg(OH)₂, MnO₂, MoO₃, Sb₂O₃, Sb₂OS₂, Sb₂Se₃, Sb₂S₃, As₂S₃, As₂Te₃, Bi₂O₂Se, Bi₂Se₃, Bi₂TeO₂, BiSbTe₃, Bi₂S₃, Bi₂Te₃, AsP, CdI₂, CdPS₃, CuS, CoPS₃, Cr₂Ge₂Te₆, Cr₂S₃, CrBr₃, CrCl₃, CrGeTe₃, CrPS₄, CrSeBr, CuCrP₂S₆, CuIn₇Se₁₁, FeCl₂, FePS₃, FePSe₃, MoTe₂, GaGeTe, GalnS₃, GaSeTe, GaSSe, GaPS₄, GaSTe, GeAs, GeSe, GeS, GeS₂, GeTe, HfSe₂, HfS₂, HfTe₂, In₂S₃, In₂Se₃, InSe, InTe, InGaSe₂, InSeBr, InSnSe, MnPS₃, MnPSe₃, MoSSe, MoWSe₂, MoWS₂, MoWTe₂, MoNbSe₂, MoO_{2.5}Cl_{0.5}, MoReS₂, MoTaSe₂, MoVSe₂, Na₂Co₂TeO₆, Nb₂SiTe₄, NbReS₂, NbReSe₂, NbS₃, Ni₂SiTe₄, Ni₃TeO₆, NiCl₂, NiI₂, NiPS₃, PbI₂, PbTe, PtTe₂, ReMoS₂, ReNbS₂, ReNbSe₂, ReSSe, SbAsS₃, SbSe, SbSI, SiP, SnPSe₃, SnS, Ta₂NiS₅, TaS₂, TaS₃, TaSe₂, TaWSe₂, TlSe, TiBr₃, SnTe₂, TiS₃, TIGaS₂, TlGaSe₂, TIGaTe₂, TlInS₂, WTe₂, WSSe, WNbSe₂, WReSe₂, ZrS₂, ZnIn₂S₄, ZnPS₃, ZnPSe₃, ZrGeTe₄, ZrS₃, ZrSe₂, ZrSe₃, ZrTe₂, ZrTe₃, Cr₂Si₂Te₆, Cr₂Te₃, CrI₃, CrSBr, CrTe₂, Fe₃GeTe₂, Fe₄GeTe₂, TaCo₂Te₂, VS₂, VSe₂, VTe₂, BiSbTeSe, BiTe, CuFeTe, HfTe₅, FeSe, FeTeSe, FeTe, NbS₂, NbSe₂, NbTe₂, NbTe₄, NiTe₂, PdBi₂, PdTe₂, SnTaS₂, TaTe₂, TiTe₂, Tl₂Ba₂CaCu₂Og, ZrSiS, CdAs₂, CuSi₂P₃, NbAs₂, PbTaSe₂, Ta₂NiSes, Ta₂NiTes, Ta₂Se₈I, TaNi₂Te₃, TiS₂, TiSe₂, WNbTe₂, ZnAs₂, ZrTe₅, LaTe₂, NbSe₃, BizSeTe₂, Bi₂Te₂S, BiInTe₃, Bi₂Se_{1.5} Te_{1.5}, Bi₄Te_{1.5}S_{1.5}, GeBi₂Te₄, PbBi₂Te₄, SnBi₄Te₇, SnSb₂Te₄, NiTe, SbTe, SiTe₂, BiTeI, InSSe, PbSnS₂, TlGaS₃, C₃N₄, Cu₂Te, GeSeTe, MnTe, As₂Se₃, CrPS₃, SnSe, WReS₂, TiBr, BaTiS₃, Al₂O₃, BiFeO₃, Ag₃AsS₃, HgS, bismuth strontium calcium copper oxide, black arsenic, or black phosphorus.

For the liquid lens 1 to function normally, it is necessary that the liquid media 5 and 6 freeze at a maximally low achievable (possible) temperature. The crystallization temperature of such liquid lens 1 should be in the range from −20 to −50 degrees Celsius, preferably −35 degrees Celsius. This is achieved by using the media with low crystallization temperature, for example, silicone oil having the freezing temperature below −60° or a mixture of water (33.3%) and glycerin (66.7%) with the freezing temperature as low as −46.5°.

The suggested liquid lens 1 operates as follows.

When no voltage is applied to the electrodes 3, and they do not form electromagnetic field FIG. 1, the liquid lens 1 is in its initial state (meniscus 4 is flat) and has a null or near null optical power, i.e. operates as a plane-parallel plate and do not change the path of the rays. When a voltage is applied FIG. 2 to the electrodes 3 and they are used to form electromagnetic field, the electroconductive liquid medium (or, alternatively, a medium with magnetic nanoparticles) under the influence of the electric field (or, alternatively, magnetic field formed by alternating voltage on the electrodes 3) moves to the periphery of the liquid lens 1, changing the meniscus 4 radius of curvature to the value R depending on the linear dimensions of the capsule 2, including the thickness and the light diameter (aperture). Hereupon, the liquid lens 1 optical power changes: the higher is the voltage applied to the electrodes 3 and, accordingly, the greater is the energy of the electromagnetic field they form, the smaller (quantitatively) is the curvature radius R and the greater is the liquid lens 1 optical power.

The maximum limit of the liquid lens 1 optical power results from the difference between the refractive indices of the liquid media 5-6 and the minimum achievable curvature radius R of the meniscus 4. In air, for the capsule 2 with the diameter d=2 mm and thickness H=1 mm (i.e. for the meniscus 4 curvature radius R=1 mm) and the refractive index difference Δn=1.2, the liquid lens 1 optical power comprises 1200 diopters. In the context of this application, here and below the liquid lens 1 optical power is understood as its maximum achievable value in air with the maximum voltage applied on the electrodes 3 and corresponding maximum electromagnetic field energy.

Such expansion of the range of achievable optical power (from 0 to 1000 diopters and more) results in unexpected technical effect—significant expansion of the liquid lens possible application areas, in particular, when the suggested high power liquid lens 1 is integrated into a wearable optical device such as a contact lens or an intraocular lens to form a magnified image of the objects in the field of view, to form augmented, virtual, or extended reality (AR/VR/XR), for prevention and treatment of myopia.

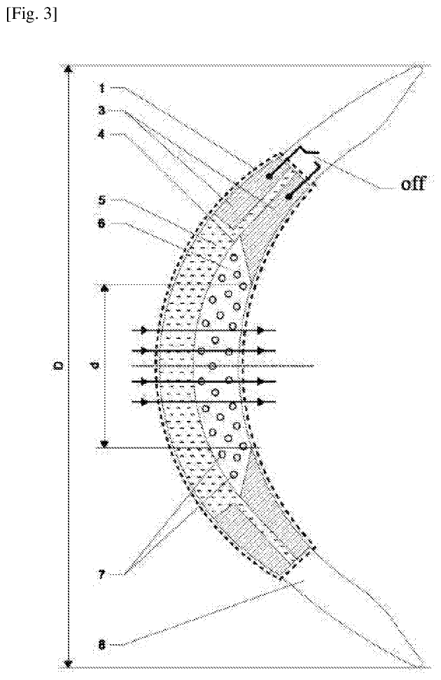

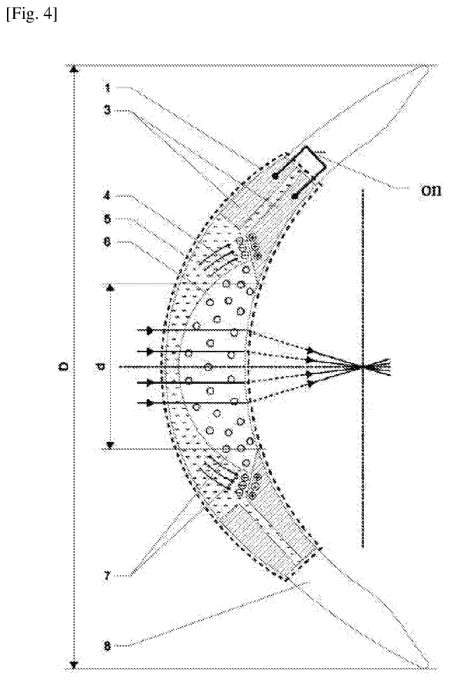

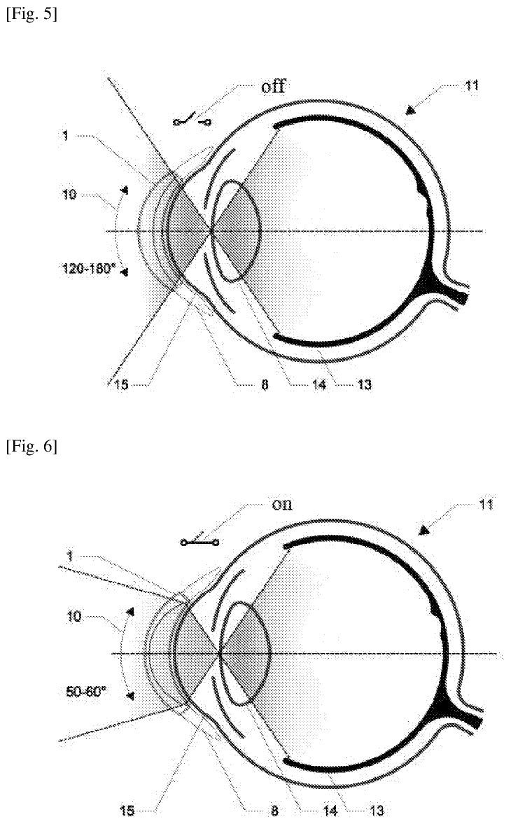

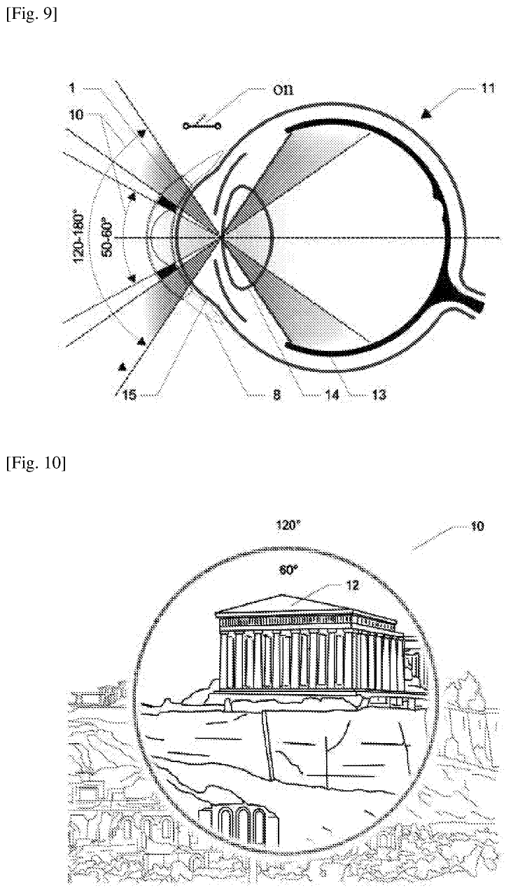

According to the first embodiment FIG. 3-FIG. 10 realizing the zoom function, the contact lens 8 only contains the power and control module 9 and a reconfigurable optical system in the form of the suggested liquid lens 1 that occupies at least a part (preferably from 30 to 60%) of the field of view 10 of the user's eye 11. The electrodes 3 of the liquid lens 1 are connected to the power and control module 9. The liquid lens 1 is adapted to focus the images of the respective objects 12 not on the retina 13 of the eye 11, but on the crystalline lens 14, to realize so called Maxwellian view (see ‘The Maxwellian view’, Gerald Westheimer, Vision Research, vol. 6, issues 11-12, Dec. 1966, p. 669-682, doi: 10.1016/0042-6989(66)90078-2). Such scheme forms a sharp magnified image on the retina 13, i.e. a visible magnification of the objects 12 occurs. The advantage of the Maxwellian view is a wide field of view (FOV) and a possibility to work with an eye having such defects as shortsightedness/farsightedness, as no vision correction is needed. With the liquid lens 1 optical power being not less than 300 diopters, it is possible to obtain two or more times magnification of the objects 12.

FIG. 3-FIG. 5 show the contact lens 8 with the built-in liquid lens 1 occupying the whole field of view 10 (100% coverage).

When no voltage is applied to the electrodes 3, and they do not form electromagnetic field, i.e. the liquid lens 1 is deactivated, the user's eye 11 has a standard vertical field of view 10 approximately 60° (double angle) 120°. In this case, the rays hitting the cornea 15 at the angles exceeding 60° in the vertical plane (for example, 70°), after passing the eye 11 optical system do not hit the retina 13 and, accordingly, cannot be perceived by the user.

When a voltage is applied to the electrodes 3 and they are used to form electromagnetic field, i.e. the liquid lens 1 is activated, it becomes an optical element with a specific optical power (250-500 diopters). In this case, due to the presence of the liquid lens 1, the rays hitting the contact lens 8 from distant objects ‘at infinity’ (the angle with the eye optical axis is 0°) focus not on the retina 13, but on the crystalline lens 14 (the crystalline lens in this case being in a relaxed state). These rays due to the Maxwellian view effect will form a sharp magnified image on the retina 13. Herewith, the rays from the closer objects 12 coming at certain angles to the optical axis (for example, up to 25°—a half-angle) focus on the crystalline lens 14 as well, hit the retina 13 as a sharp image, and are pictured by the eye optical system. The rays exceeding a critical angle (for example, greater than) 25° focus in front of the crystalline lens 14 due to a high optical power of the liquid lens 1 and either hit the very edge of the retina 13 in a defocused state or miss the retina 13 at all and, accordingly, are not visible for the user.

A two-three times (preferably 2.4×) optical magnification within the whole field of view area 10 (100% coverage) can be achieved with the following liquid lens 1 parameters:

| | |
|---|---|
| diameter | d = 9-11 mm (preferably 10 mm); |
| thickness | H = 1-2 mm (preferably 1.6 mm); |
| medium 5 refractive index | n₁ = 1.2-1.33 (preferably 1.2); |
| medium 6 refractive index | n₂ = 1.7-3 (preferably 3); |
| capsule 2 radius | R₁ = 6.5-7.5 mm (preferably 7 mm, |
| of curvature | corresponds to the cornea 15 radius of a |
| | specific eye 11); |
| meniscus 4 radius | R = 5-R₁ mm (preferably R = 5.25-R₁ mm); |
| of curvature | |
| optical power | 250-500 dpt (preferably 340 dpt). |

The zoom effect based on the operation of the liquid lens 1 in the contact lens 8 is stepwise, not pancratic, i.e. in fact there are two operating end positions that matter—the liquid lens 1 is deactivated (the field of view is focused on the retina 13) and the liquid lens 1 is activated (the field of view is focused on the crystalline lens 14). All of the intermediate states when the liquid lens 1 optical power is varied to focus in the area between the crystalline lens 14 and the retina 13 are nonoperating as in such cases the eye 11 is unable to build a focused image on the retina 13.

Nevertheless, the liquid lens 1 optical power controlling can be used for another purpose. For example, in the above example the crystalline lens 14 was in a relaxed state, i.e. was focused at infinity. During the eye accommodation, the crystalline lens 14 back wall changes its radius of curvature and the optical power. This also means that the rays, which previously were focused by the liquid lens 1 onto the crystalline lens 14, now can come to the retina 13 in a slightly defocused state as the crystalline lens 14 back wall has changed its position. By decreasing the liquid lens 1 optical power in accordance with the increased optical power of the crystalline lens 14, it is possible to compensate the change made to the optical system by the crystalline lens 14 and once again achieve a clear and sharp image on the retina as a result of the eye accommodation. Thus, if an image magnified using the zoom function is blurred as a result of the eye accommodation, then, by controlling the meniscus 4 curvature, it is possible to compensate this blurring and to make the magnified image clear and sharp again.

The suggested contact lens 8 according to the first embodiment is not able to implement the zoom function within the whole field of view 10, but only within a part of it: for example, 50% of the eye's central field of view-such embodiment will have more compact dimensions. FIG. 7 shows such contact lens 8 with the built-in liquid lens 1 when no voltage is applied to the electrodes 3, i.e. in the case when the liquid lens 1 has no optical power and operates as a plane-parallel plate. Herewith, the eye 11 has a standard vertical field of view of ~60° (double angle) 120°, and the rays hitting the cornea 15 at greater angles do not come to retina 13 after passing the eye optical system and, accordingly, cannot be pictured by the user's eye.

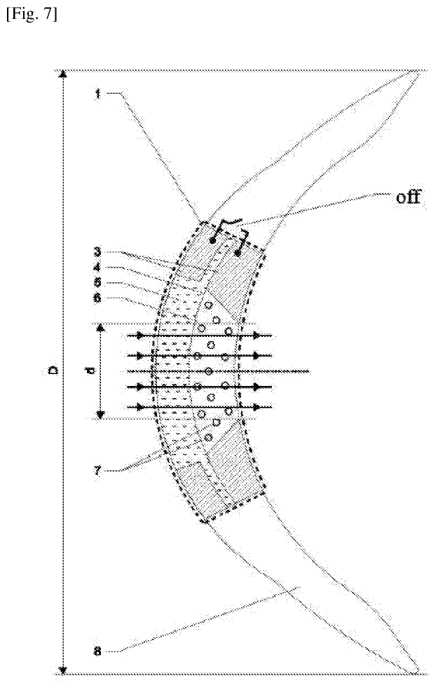

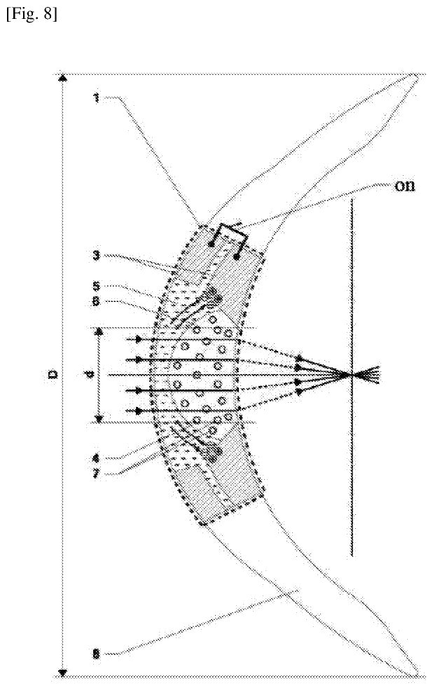

FIG. 8 shows the same contact lens 8 with a voltage applied to the electrodes 3, i.e. in the case when the liquid lens 1 has an optical power defined by the curvature radius of the meniscus 4 (which is formed due to the presence of electromagnetic field) and by the refractive indices n₁ and n₂. In this case, the rays hitting the contact lens 8 part with the liquid lens 1 from infinity (angle 0°) are focused not on the retina 13, but on the crystalline lens 14, which at this time is in a relaxed state. Due to the Maxwellian view effect, these rays will form a sharp image on the retina 13. Likewise, all the rays hitting the liquid lens 1 at certain angles (for example, up to 25°—a half-angle) focus on the crystalline lens 14 as well and hit the retina 13 in a sharp state, and are pictured by the eye 11 optical system. The rays that come to the liquid lens 1 at greater angles (for example, greater than 25°) focus in front of the crystalline lens 14 due to a high optical power of the liquid lens 1. Thus, they either hit the very edge of the retina 13 in a defocused state or miss the retina 13 at all and, accordingly, cannot be pictured by the user's eye 11.

Additionally, there are the rays that hit the contact lens 8 peripheral part that is not covered by the liquid lens 1. These rays pass through the contact lens 8 unchanged (if the contact lens 8 itself has 0 diopters) and are focused by the crystalline lens 14 on the retina 13 in a regular manner. Thus, within the field of view 10, a central part is formed with a magnified image of the objects 12 and with a reduced angle of view (for example, from 120° to 50°—full vertical angle), as well as a normal peripheral part of the angle of view containing the image of the objects 12 with regular size FIG. 10.

To realize a two-three times optical magnification (preferably 2.4×) within a 50% area of the central field of view, the liquid lens 1 should have the following parameters:

| | |
|---|---|
| diameter | d = 4-6 mm (preferably 5 mm); |
| thickness | H = 0.3-0.5 mm (preferably 0.35 mm); |
| medium 5 refractive index | n₁ = 1.2-1.33 (preferably 1.2); |
| medium 6 refractive index | n₂ = 1.7-3 (preferably 3); |
| capsule 2 radius | R₁ = 6.5-7.5 mm (preferably 7 mm, |
| of curvature | corresponds to the cornea 15 radius of a |
| | specific eye 11); |
| meniscus 4 radius | R = 4-R₁ mm (preferably R = 4.3-R₁ mm); |
| of curvature | |
| optical power | 250-500 dpt (preferably 420 dpt) |

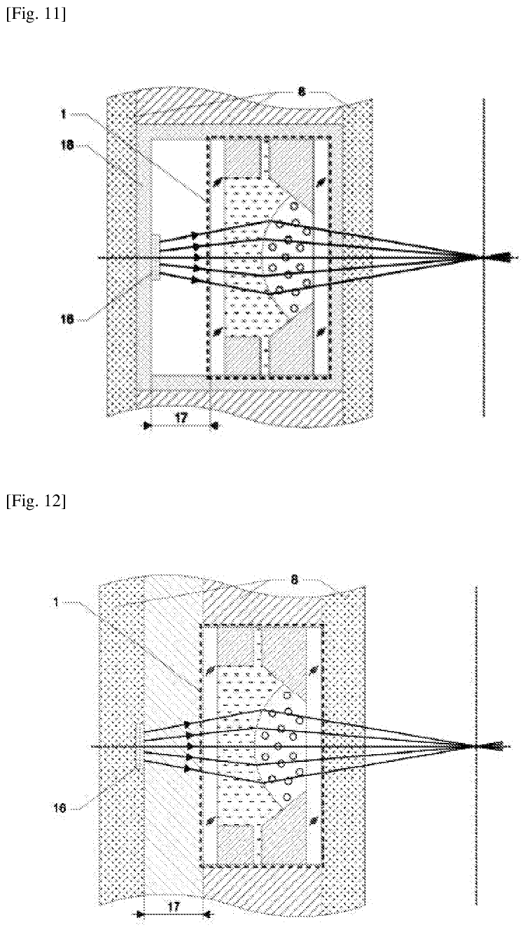

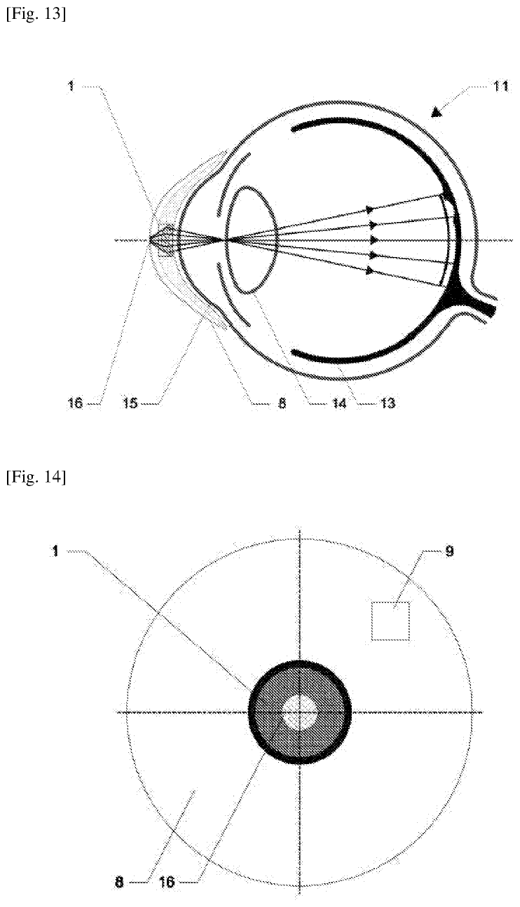

According to the second embodiment FIG. 11-FIG. 14 realizing the AR/VR/XR function, the contact lens 8 comprises the display 16, the power and control module 9, and the optical system in the form of the liquid lens 1 disposed between the display 16 and the eye 11. The electrodes 3 of the liquid lens 1 are connected to the power and control module 9, and the liquid lens optical power comprises at least 800 diopters. In this case, the liquid lens 1 enables focusing the emission from the display 16 screen on the eye-crystalline lens 14 of the user's eye 11 to realize the Maxwellian view, as the distance from the source of the image (focal plane) to the liquid lens 1 is much less than the distance from the liquid lens 1 to the retina 13 (image plane). Such scheme makes it possible to form a clear image of the display 16 on the retina 13 without aberrations, with high transmittance and wide field of view, and laid over the real scene.

The image source represents the display 16 based on a light emitting diode matrix (LED, microLED, OLED, and the like). The display 16 can have the size from 50×50 to 500×500 μm, preferably 100×100 μm, with the pixel size up to 1 μm. Alternatively, the display can have any other shape, such as rectangular or round with the diameter of 50, 100, 500 μm and with the pixels arranged in a honeycomb pattern (hexagons). In this case, the display 16 size, as referred to in this application, is understood as the largest linear dimension: a diagonal line of a square or rectangle, a circle diameter, a distance between the opposite vertexes of a hexagon, etc.

The display 16 is attached to the external surface of the contact lens 8 and directed towards the pupil of the user's eye 11. The display 16 is powered from the power and control module 9, which may contain a rechargeable battery and/or inductance coil built into the contact lens 8.

To ensure the display 16 resides in or near the focal plane, the distance between the display and the liquid lens 1 along the optical axis should be 50-250 μm, preferably 100 μm. The corresponding gap 17 can be formed by the hollow chamber 18 filled with air (n=1) or by a layer of polymer material (PMMA or another substance that is transparent in the visible range of the spectrum).

The embodiment with the air gap 17 is shown in FIG. 11. In this case, the whole projecting optical system represents a hollow chamber 18 made of plastic, polymer, glass, crystal, nano-glass ceramics, or another material that is transparent in the visible range of the spectrum, containing the display 16 installed on its one side and the liquid lens 1 on the other. In this case, the gap 17 between the display 16 and the liquid lens 1 is filled with air or another gas. Such projector structure is installed into the contact lens 8 as follows: onto the contact lens 8 primary polymer layer (closest to the cornea 15), in the center, the chamber 18 is installed together with the display 16 and the liquid lens 1, then, on the periphery thereof, all the required electronics is installed (the power and control module 9 with the inductance coil, rechargeable battery, power conductors, and the control electrodes 3 of the liquid lens 1), the periphery is covered with a polymer and polymerized to fix the chamber 18, following which a finishing polymer layer is applied (furthest from the cornea 15). For the liquid lens 1 having the light diameter (aperture) of 160 μm and the thickness of 40 μm, the gap 17 should comprise 100 μm.

An alternative embodiment of a projecting system with the gap 17 formed by a layer of polymer material is shown in FIG. 12. Such projector structure is installed into the contact lens 8 as follows: onto the contact lens 8 primary polymer layer (closest to the cornea 15), in the center, the liquid lens 1 (that is thinner than the whole above described projecting system with the chamber 18) is installed, then, at the periphery, all the required electronics is installed (the power and control module 9 with the inductance coil, rechargeable battery, power conductors, and the control electrodes 3 of the liquid lens 1), the periphery is covered with a polymer and polymerized, following which the intermediate polymer layer of the gap 17 is applied, the display 16 is installed in the center and connected to the power and control module 9, and after that a finishing polymer layer is applied (furthest from the cornea 15). The difference in this case consists in that the air gap 17 (n=1) between the display 16 and the liquid lens 1 is replaced by the gap 17 formed by the polymer layer (n=1.2-1.4), which may negatively affect the size of the whole optical system along the optical axis—the system has to be somewhat thicker than in the previous case. Herewith, while the display 16 will have a very wide ray divergence (spatial angle 140-180 degrees) in air, inside the polymer layer the rays divergence can be significantly reduced in accordance with the Snell's law (the higher is the polymer refractive index, the lower is the divergence of the rays coming from the display 16). For example, when using a polymer having the refractive index of ~1.5 in the visible range of the spectrum, the display rays divergence can be reduced from 140-180 degrees to 75-90 degrees. Reducing the display 16 divergence angle is required in order to reduce the inlet aperture (light diameter) of the liquid lens 1 and thereby reduce its minimum overall dimensions across the optical axis. For the liquid lens 1 having the light diameter (aperture) of 500 μm and the thickness of 200 μm, the gap 17 should comprise 150 μm.

The suggested contact lens 8 according to the second embodiment operates as follows.

Upon activation of the display 16, the display screen forms a divergent beam of rays which are collected and focused by the liquid lens 2 onto the user's crystalline lens 14. This beam is focused by controlling the curvature of the meniscus 4 of the liquid lens 1 using the electromagnetic field formed by the electrodes 3. Thereby, the image aberrations are prevented, the user vision defects (shortsightedness or farsightedness) are taken into account, and a wide field of view is formed—up to 100° (full angle). Herewith, on the retina 13, a magnified sharp and clear image of the display 16 is formed.

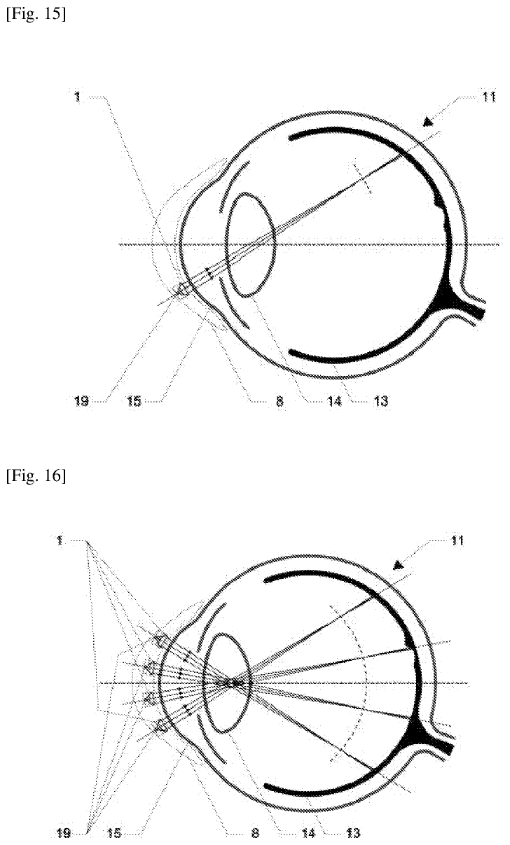

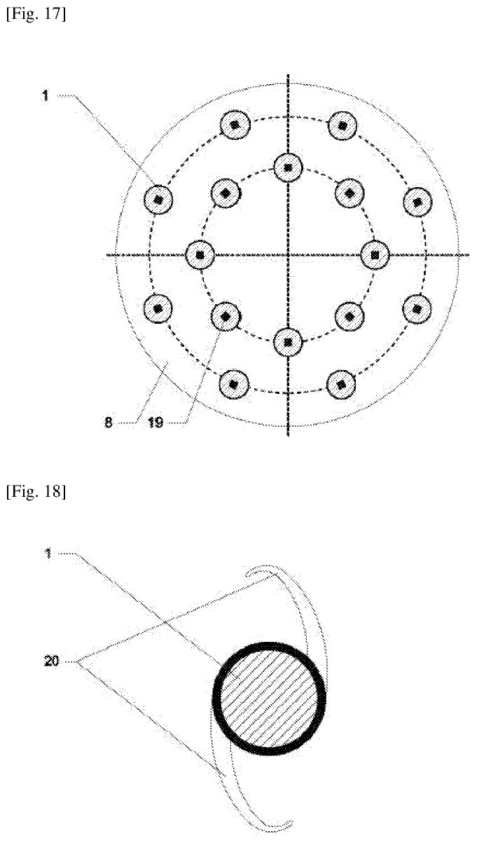

According to the third embodiment FIG. 15-FIG. 17 realizing the function of preventing and treating myopia, the contact lens 8 comprises one or several light emitting diodes 19 (for example, LED) directed towards the user's eye 11, the power and control module 9, and the optical system in the form of the liquid lens 1 disposed between each light emitting diode 19 and the eye 11. The light emitting diodes 19 and the electrodes 3 of the liquid lens 1 are powered and controlled from a similar power and control module 9, which may contain a rechargeable battery and/or an inductance coil built into the contact lens 8. To avoid overlapping with the central part of the pupil and interfering with normal vision, the light emitting diodes 19 (preferably from 2 to 40 diodes) are located in the contact lens 8 peripheral part, for example, 5-10 mm from its optical axis.

In this embodiment, the liquid lens 1 should provide the capability to focus the emission from the light emitting diode 19 in front of the retina 13 of the user's eye 11. While in operation, on a signal from the control electrodes 3, the liquid lens 1 is able to change the meniscus 4 curvature, thereby focusing the stimuli from the light emitting diodes 19 at different distances from the retina 13. Herewith, the degree of defocus of the spot of emission from the light emitting diode 19 on the retina 13 can vary in real time and should comprise from 0.5 to 10 diopters, preferably from 2 to 6 diopters. For this purpose, the liquid lens 1 optical power should be preferably not less than 1000 diopters.

The suggested contact lens 8 according to the third embodiment operates as follows.

In accordance with a control signal the light emitting diode 19 and electrodes 3 of the liquid lens 1 are periodically powered by the module 9. When using the liquid lens 1 having the light diameter (aperture) of 1 mm and the thickness of 0.2 mm, a 1×1 μm blurred image of the light emitting diode 19 located 0.7 mm from the liquid lens is formed on the retina 13. For the eye 11 with the longitudinal dimension of 24 mm (distance from cornea 15 to retina 13), the meniscus 4 radius of curvature R=1.34 mm will correspond to myopic defocus D=2 diopters, while the curvature radius R=1.33 mm will correspond to myopic defocus D=6 diopters. As the user's eye 11 can have different longitudinal dimensions, using the reconfigurable liquid lens 1 makes such contact lens 8 with the myopia prevention and treatment function very flexible in use, as, by controlling the curvature of the liquid lens 1, it is possible to set the required degree of defocus of the stimuli. Furthermore, the disclosed contact lens 8 according to the third embodiment makes it possible to significantly expand the range of possible therapeutic intervention by providing the possibility to gradually vary the degree of defocus of the light emitting diodes 19 image that is formed on the user's retina.

The disclosed intraocular device FIG. 18-FIG. 24 comprises an intraocular lens containing the liquid lens 1, the holding elements 20 to fix said intraocular lens inside the user's eye 11, and the power and control module 9. The power and control module 9 is configured to convert the user's control signals into the energy of the electromagnetic field formed by the control electrodes 3, and to vary the optical power of the liquid lens 1. Thereby, the disclosed intraocular device is able to accommodate in accordance with the user's control signals by analogy with the activity of a natural crystalline lens.

The inlet and outlet windows (walls) of the capsule 2 of the liquid lens 1, as in the previous embodiments, should be made of a material that is transparent in the visible range of the spectrum. When using as an intraocular lens, to make implantation into the eye 11 easier, the capsule 2 is preferably made of a biocompatible elastic polymer material.

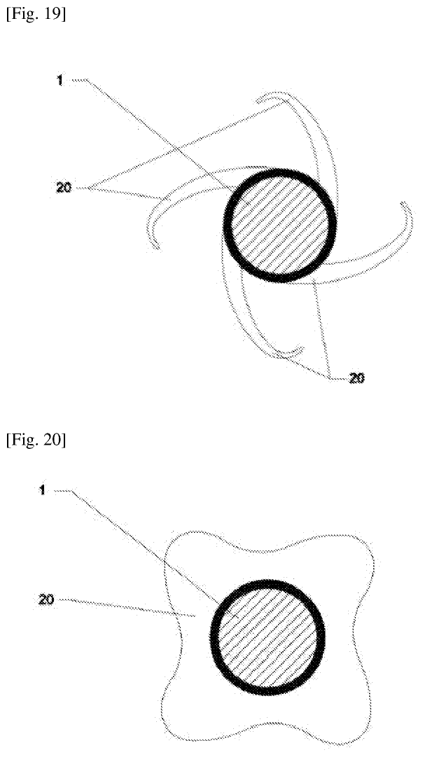

As a one of the implantation options, a surgeon, through a microincision on the cornea 15, implants the intraocular liquid lens 1, rolled into tube, into the user's eye 11. Inside the eye, the liquid lens 1 unrolls and is fixed by special holding elements 20. Said holding elements 20 can be implemented as two or more tabs independently made of elastic polymer material FIG. 18-FIG. 19 or as castings formed integrally with the capsule 2 FIG. 20. The elements 20 are positioned at 3-10 degrees (preferably 4-7 degrees) to the plane that is perpendicular to the optical axis.

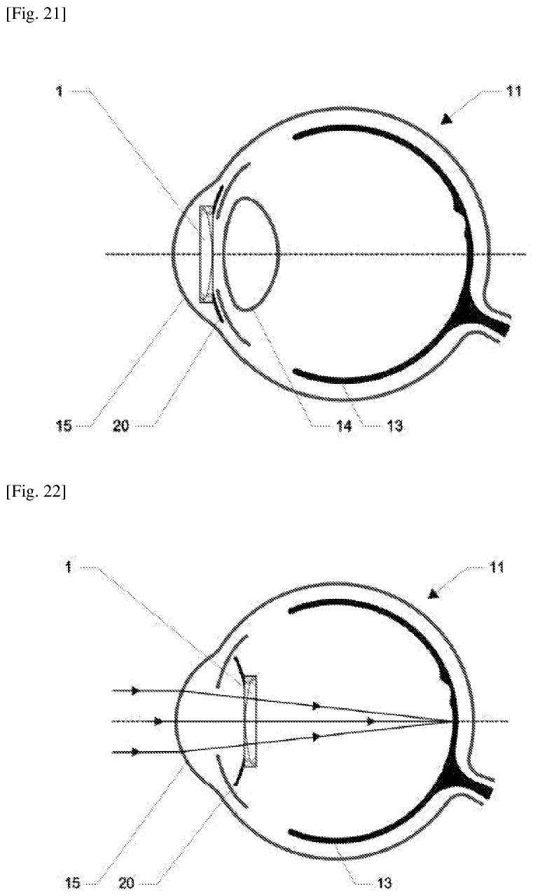

There are two options to implant the disclosed intraocular device: either in the anterior chamber or posterior chamber of the user's eye. When implanting into the anterior chamber FIG. 21, the elements 20 unfold and fix themselves between the cornea 15 and the iris. When implanting into the posterior chamber FIG. 22-FIG. 24, the elements 20 unfold and fix themselves between the iris and the ciliary body—this is a more natural position as this is the place where the removed crystalline lens was located.

The human crystalline lens optical power is known to be within the range of 19-33 diopters. The crystalline lens, being in a relaxed state, has the optical power of ~19 diopters that enables focusing the rays from distant objects (at a hypothetical infinity). To accommodate at infinity, the liquid lens 1 should also be adapted to form the optical power of 19 diopters, which corresponds to a specific curvature of the meniscus 4 FIG. 22. For the liquid lens 1 having the diameter of 10 mm, the thickness of 1 mm, the first liquid medium refractive index n₁=1.33 (standard value for water), and the second liquid medium refractive index n₂=3.0, the optical power of 19 diopters is achieved with the meniscus 4 radius of curvature R=88 mm. To form the electromagnetic field providing such curvature, the electrodes 3 should be supplied with the corresponding voltage upon a signal from the power and control module 9. In such condition, the intraocular device is an analogue of a natural crystalline lens in a relaxed state.

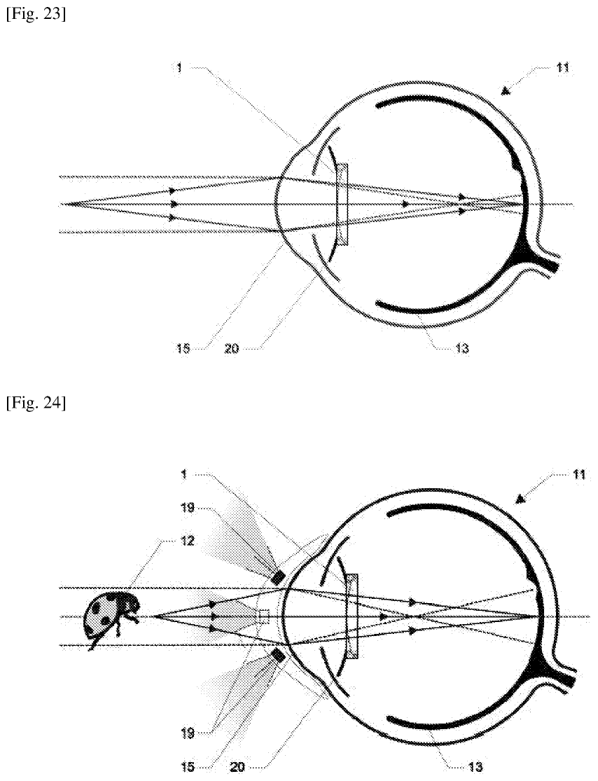

The human crystalline lens, being in a state of maximum tension, has the optical power of ~33 diopters that enables focusing the rays from the objects located nearby, in so called ‘near point’, on the retina 13. The near point is a minimum distance from the object of interest to the eye 11 (more specifically-its cornea 15) on which the eye is able to focus. This distance depends on a person, their age and health, but cannot be less than 70-80 mm. To accommodate on a near point, the liquid lens 1 in the disclosed intraocular device should have the optical power of 33 diopters as well, which corresponds to a different radius of curvature of the meniscus 4 FIG. 23. For the same liquid lens with the diameter of 10 mm, the thickness of 1 mm, with n₁=1.33 and n₂=3.0, the optical power of 33 diopters is achieved at the meniscus 4 radius of curvature R=51 mm. To provide such curvature of the meniscus 4, the electrodes 3 should form the electromagnetic field with the energy higher than that for 19 diopters, i.e. the voltage on the electrodes should be higher.

Despite the fact that a human's ordinary crystalline lens is physically unable to focus on a point located closer than 70-80 from the cornea of the eye, the disclosed intraocular device is able to do so as the liquid lens 1 range of adjustment is not yet used up. Thus, the liquid lens 1 can vary its optical power to higher values up to 200 diopters, which means it can focus on a closer point. For example, the same liquid lens with the diameter of 10 mm, the thickness of 1 mm, with n₁=1.33, n₂=3.0, and the optical power of 128 diopters, with the meniscus 4 radius of curvature R=13 mm enables focusing on the object 12 at the distance of 8.2 mm from the cornea 15 FIG. 24.

Thus, the disclosed intraocular device expands the human eye capabilities and makes it possible to examine at objects from a very close distance, in fact being an analogue of a macro-lens in photography or a microscope lens with a specific magnification. Herewith, the angular resolution (which comprises 1-1.5 degrees in the center area of a healthy human eye) will depend on the liquid lens 1 surface quality, capsule 2 surface curvature, and other conditions.

When examining at the objects 12 from such ultra-close distance, especially when they are nontransparent, problems may arise due to low brightness of such objects 12. Eyebrows, brow ridges, eyelashes, cheeks, nose, and other parts of the user's head may block the natural light from the environment from falling onto the object 12 located at an ultra-close distance and prevent reflecting the scattered light from the objects 12 into the eye 11. This problem can be solved, for example, by using the contact lens 8 with one or several built-in light emitting diodes 19 directed away from the eye 11. Such light emitting diodes 19 will brighten the objects 12 at the ultra-close distance and can be connected to a similar power and control module 9, which can contain a rechargeable battery and/or inductance coil, solar battery, asic-chip, etc. built into the contact lens 8. The light emitting diodes 19 are preferably disposed on the periphery of the contact lens 8 outside the eye's field of view and outside the pupil diameter, for example, at the radius of 6-9 mm from the lens optical axis. In this embodiment, the power and control module 9 of the liquid lens 1 should be configured to send an activation signal to the light emitting diodes 19 (via the power and control module 9 of the contact lens 8) when the liquid lens 1 optical power has exceeded 35 diopters.

The disclosed intraocular device operates as follows.

During the eye accommodation on external objects 12, the human brain sends a signal in the form of an electric signal transmitted via the nerve knots to the eye 11 ciliary muscle forcing the muscle to contract and relax (when the crystalline lens is removed, the ciliary muscle remains in place). In an attempt to focus, the ciliary muscle contracts, and this user's control signal via the power and control module 9 of the liquid lens 1 is converted into the energy of the electromagnetic field formed by the electrodes 3 (for example, by converting the ciliary muscle pressure into an electric signal using a piezoelectric element integrated into the tab 20 and an amplitude amplifier that multiplies said signal from the piezoelectric element). The change in the electromagnetic field energy results in a change in the meniscus 4 curvature and, accordingly, in the liquid lens 1 optical power. When the liquid lens 1 optical power exceeds 35 diopters, the light emitting diodes 19 turn on automatically to brighten the object 12.

Thus, using the disclosed liquid lens containing a colloidal solution of nanoparticles and, by this reason, having an increased difference in refractive indices of the liquid media and, hence, increased range of achievable optical power, significantly expands the lens possible application areas and enables implementation of the disclosed embodiments of the contact lenses and intraocular device.

1. A liquid lens implemented as a capsule containing an immiscible first liquid medium having a refractive index n1 and a second liquid medium having a refractive index n₂, such that n₂ is greater than n₁, and control electrodes, wherein one of the first liquid medium and second liquid medium is adapted to be controlled by an electromagnetic field via the control electrodes, and wherein the second liquid medium represents a colloidal system formed by a liquid and solid high-refractive nanoparticles whose refractive index is higher than the refractive index of the liquid, wherein said first liquid medium and second liquid medium are preferably implemented in such a way that the difference of their refractive indices greater than 1.5.

2. The liquid lens according to claim 1, wherein the solid high-refractive nanoparticles comprise at least one of TiO₂, GaP, AlSb, GaSb, InSb, AlAs, GaAs, InAs, SiC, TiC, VC, Fe₂O₃, InP, PbSe, ZnSe, CdS, CuGaS₂, CdTe, Te, ZnTe, Bi₄Ti₃O₁₂, and PbTiO₃.

3. The liquid lens according to claim 1, wherein the solid high-refractive nanoparticles are made of a van der Waals material comprising two-dimensional layers bonded with each other by van der Waals forces.

4. The liquid lens according to claim 3, wherein the van der Waals material is at least one of MoS₂, MoSe₂, WSe₂, Cd₃As₂, Mo₂Ga₂C, Nb₂AlC, Ti₂AlC, SnS₂, SnSe₂, ReS₂, ReSe₂, GaSe, PdS₂, PdSe₂, PtS₂, PtSe₂, and GaS.

5. A contact lens containing a power and control module and a reconfigurable optical system, wherein the optical system is equipped with the liquid lens of claim 1 occupying at least a part of an eye field of view of a user and adapted to focus images of the objects located within the part of the field of view on an eye lens of the user, and to provide visible magnification of the objects.

6. The contact lens according to claim 5, wherein the liquid lens has an optical power of not less than 300 diopters and wherein the optical power enables magnification of the objects within the field of view of the user of at least two times.

7. The contact lens according to claim 5, wherein the liquid lens comprises from 30 to 60% of the part of the field of view of the user.

8. A contact lens containing a display with a screen directed towards an eye of the user, a power and control module, and an optical system disposed between the display and the eye of the user, wherein the optical system is equipped with the liquid lens of claim 1 configured to focus an emission from the screen on an eye lens of the user.

9. The contact lens according to claim 8, wherein the optical power of the liquid lens of claim 1 comprises at least 800 diopters.

10. The contact lens according to claim 8, wherein the liquid lens of claim 1 is installed with a gap relative to the screen.

11. The contact lens according to claim 10, wherein the gap is formed by a hollow chamber.

12. The contact lens according to claim 10, wherein the gap is formed by a layer of polymer material.

13. A contact lens containing at least one light emitting diode disposed in a peripheral part of the contact lens and directed towards an eye of the user, a power and control module, and an optical system disposed between the light emitting diode and the eye of the user, wherein the optical system comprises the liquid lens of claim 1 configured to focus an emission from the light emitting diode in front of a retina of the user.

14. The contact lens according to claim 13, wherein the optical power of the liquid lens comprises at least 1000 diopters.

15. The contact lens according to claim 13, wherein the liquid lens is adapted to vary a degree of defocus of the emission from the light emitting diode on the retina of the user in a range of from 0.5 to 10 diopters.

16. The contact lens according to claim 13, wherein the contact lens comprises from 2 to 40 light emitting diodes disposed in the peripheral part of the contact lens.

17. An intraocular device comprising an intraocular lens; adjustable in accordance with control signals input by a user and fixation elements to fix the intraocular lens inside an eye of the user, wherein the intraocular lens is equipped with the liquid lens of claim 1 and a power and control module configured to convert the control signals input by the user into an energy of an electromagnetic field formed by control electrodes of the liquid lens.

18. The intraocular device according to claim 17, wherein the power and control module is configured to vary an optical power of the liquid lens at least from 19 to 33 diopters.

19. The intraocular device according to claim 18, wherein the power and control module is configured to vary the optical power of the liquid lens up to 200 diopters.

20. The intraocular device according to claim 19, wherein the power and control module is configured to transmit an activation signal to light emitting diodes in a contact lens of the user when the liquid lens optical power exceeds 35 diopters.

21-23. (canceled)
